(19) United States
(12) Patent Application Publication
(10) Pub. No.: US 2005/0090792 A1
(43) Pub. Date: Apr. 28, 2005

(54) Two-layered pantiliner with wings and three-layered pantiliner with wings

(76) Inventor: Karen Yvonne Johnston, Pocatello, ID (US)

Correspondence Address: Dr. Karen Johnston, 6375 S. Bannock Hwy, Pocatello, ID, 83204, US

(21) Appl. No.: 10/703,226

(22) Filed: Oct. 24, 2003

(51) Int. Cl. A61F 13/15; A61F 13/20

(52) U.S. Cl. 604/385.04

(57) ABSTRACT

My invention is a Two-Layered Pantiliner with Wings and a Three-Layered Pantiliner with Wings that have multiple layers of separate, removable and disposable pads adhered together that can be removed as needed ensuring continued protection. The trademark names are “Double” and “Triples”. The regular version of the Two-Layered Pantiliner has a bottom layer that measures 7¼″ and a top layer that measures 7″ approximately. The regular version of the Three-Layered Pantiliner has a bottom (third) layer that measures 7¼″, the middle (second) layer measures 7″, and the top (first) layer measures 6¾″ approximately. A shorter version of both pantiliners has similar measurements beginning with a 5¾″ bottom layer with ½″ increments(s) for the upper layer(s). The longer version has similar measurements beginning with 9¼″ for the bottom layer with ½″ increment(s) for the upper layer(s). By having multiple layers, rather than the customary one layer of protection, the wearer is able to remove the soiled top layer, leaving another layer underneath (and a third layer on the Three-Layered version) for continued protection of her clothing.

### BACKGROUND OF THE INVENTION

Many American women wear pantiliners to achieve continual protection and freedom from embarrassing odors throughout the day. It is often inconvenient to carry an extra pantiliner from the work area to the restroom.

I created a multi-layered pantiliner with wings to wear away from home. The wearer can discreetly remove the used top layer of pantiliner and still have a pantiliner remaining on her underclothing. The three-layered pantiliner allows this to be done a second time, leaving the third layer.

The need for such an invention exists because the standards of personal cleanliness demand it. Women fear offending others by smelling unclean; therefore they use pantiliners to insure cleanliness of their clothing. The women who currently use the single pantiliners currently on the market will be enthusiastic and faithful consumers of this product because it is a vast improvement of the existing pantiliners. Once consumers are familiar with the product, its convenience will make it highly marketable and the multi-layered pantiliners will become an essential part of their daily grooming.

### BRIEF SUMMARY OF THE INVENTION

All pantiliners on the market today are merely single pads. The Two- and Three-Layered Pantiliners with Wings enables the wearer to discreetly remove the soiled top pad(s) and still have a clean pad remaining on her underclothing, discreetly and simply. If the wearer chooses the three-layered pad with wings, the soiled top layer of the pad can be removed again later as needed, leaving the third clean layer. The wearer will not have the burden of carrying extra pads with her which can be embarrassing in many situations and she will not face the inconvenience of running out of pads away from home. Two or three layers work very well for a full day at work or in other situations away from home.

### BRIEF DESCRIPTION OF THE DRAWINGS

The attached drawings show the Two-Layered Pantiliner with Wings and the Three-Layered Pantiliner with Wings as two and three separate pads, respectively, adhered together to form one pantiliner. The adhesive between the layers is light so it is easily released from the pad underneath. The bottom pad has a slightly stronger adhesive protected by a strip of paper because it adheres to the fabric of the underclothing.

The bottom pad has small adhesive flaps or “wings” attached to the center on each side so the wearer can fold the adhesive areas around the fabric of the crotch of the underclothing for extra security of staying in place.

The pads increase in size from top to bottom to enable the user to easily separate one layer from another. On the two-layered pad, the top layer measures about 7″, and the bottom layer measures about 7¼″. On the three-layered pad, the top layer measures about 6¾″, the middle layer measures about 7″, and the bottom layer measures about 7¼″. (Longer or shorter versions can easily be made by adding or subtracting two or three inches to these measurements.) The pads are approximately 3″ wide. The attached drawings show the following views of the invention:

Two-Layered Pantiliner with Wings:

FIG. 1, the top view showing the location of the wings and the graduated lengths of the two pads;

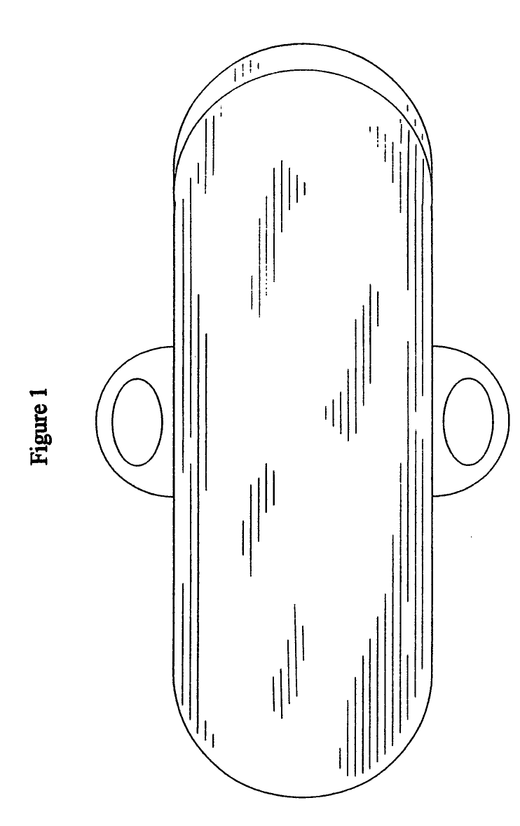

FIG. 2, a fully-assembled side view showing two pads adhered together with the graduated lengths of the two pads;

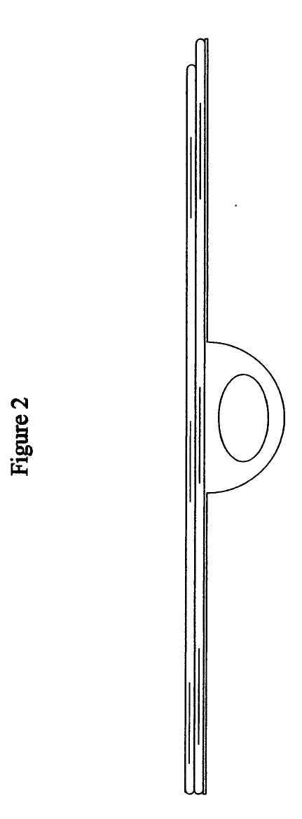

FIG. 3, an exploded side view showing thepads separated and the paper base attached to the adhesive on the bottom pad. Adhesive used between the pads is not shown but is present, as is adhesive between layers.

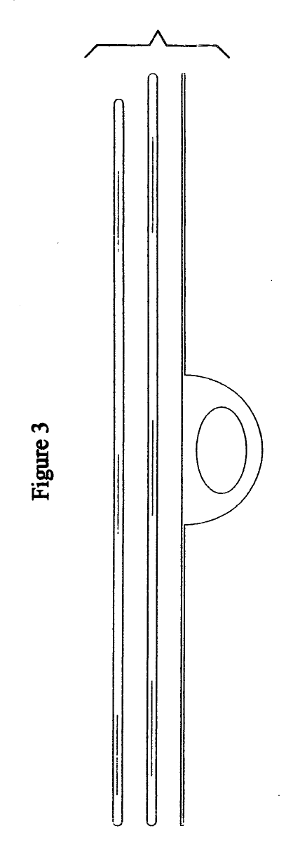

FIG. 4, the bottom view showing the paper strip over the adhesive down the center length of the pad and two small paper covers over the adhesive on the wings.

Three-Layered Pantiliner with Wings:

FIG. 5, the top view showing the location of the wings and the graduated lengths of the three pads;

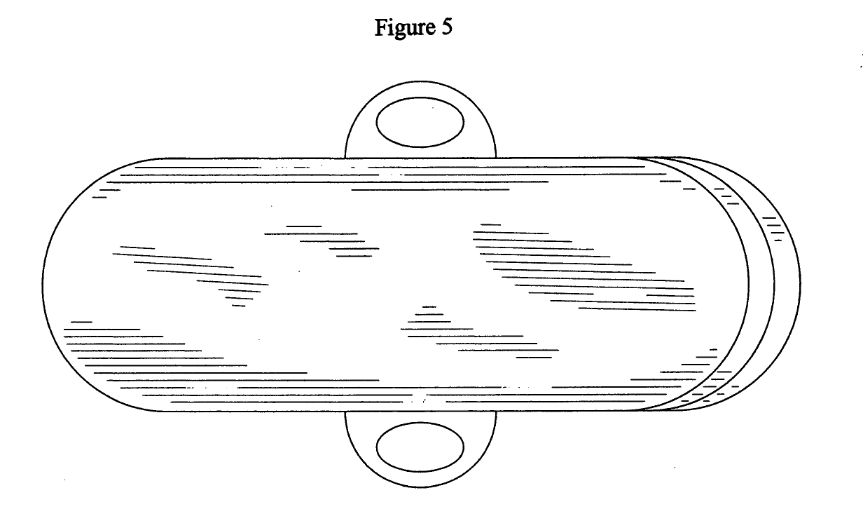

FIG. 6, a fully-assembled side view showing three pads adhered together with the graduated lengths of the three pads;

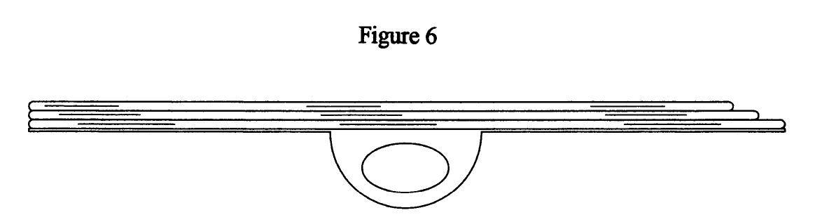

FIG. 7, an exploded side view showing the pads separated and the paper base attached to the adhesive on the bottom pad. Adhesive used between pads one and two is not shown but is present, as is adhesive between layers two and three;

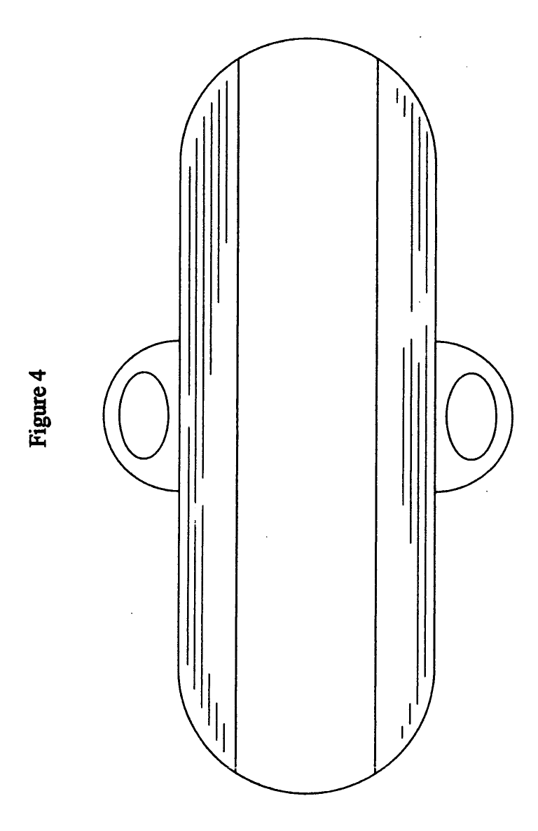

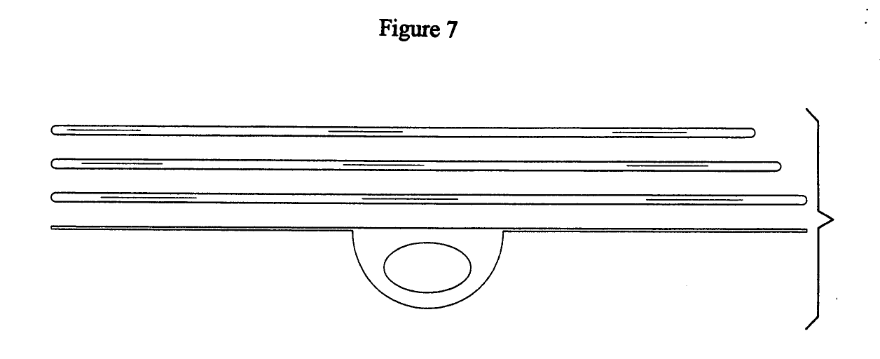

FIG. 4 is the same for both the two-layered and the three-layered pantiliners.

### DETAILED DESCRIPTION OF THE INVENTION

My invention is a Two-Layered Pantiliner with Wings and a modification of the invention, a Three-Layered Pantiliner with Wings. The pantiliners have separate pads adhered together and easily separated for two or three uses, thereby ensuring a woman's continual cleanliness throughout the day.

Because there are multiple layers, the wearer is able to remove the soiled pad two or even three times throughout the day or at intervals as needed, leaving a new layer underneath to continue protecting the clothing.

Fear of unpleasant odors and the inconvenience of carrying extra pads around are nicely eradicated by using a multiple-layer pad.

The pantiliners are made of the usual absorbent cotton and other absorbent materials used for feminine sanitary pads. Its uniqueness is that there are two or three layers instead of one, providing convenience and continual cleanliness for the wearer. The bottom layer has adhesive material between the pad and the paper to keep the adhesive from sticking to other pads in the pack. In addition, there is another layer of adhesive between layers one and two (and between layers two and three in the three-layered pantiliner) to adhere the pads together until the wearer removes the upper pad.

1. This multi-layered pantiliner has separate pads that are adhered together to form one pantiliner. The adhesive on the top layers is light but sufficient to adhere to the pad(s) beneath. The bottom pad has a slightly stronger adhesive and a strip of removable paper attached to the pad and to the smaller strips of adhesive paper on the wings to enable the pad to be firmly attached to underclothing.
This two and three-layered concept enables the user to dispose of the soiled layer by peeling it off and discarding it, leaving one or two more clean layers. On the three-layered pantiliner, the second layer can also be removed, leaving a clean third layer, thus ensuring continued cleanliness throughout the day.
The undersigned, being the inventor of the disclosed invention, requests that the enclosed papers are accepted under the Disclosure Document Program, and that they be preserved for a period of two years.
